(19) United States
(12) Patent Application Publication
(10) Pub. No.: US 2014/0271468 A1
(43) Pub. Date: Sep. 18, 2014

(54) ASTHMA IMAGING

(71) Applicant: Purdue Research Foundation, West Lafayette, IN (US)

(72) Inventors: Philip S. Low, West Lafayette, IN (US); Jiayin Shen, West Lafayette, IN (US)

(73) Assignee: Purdue Research Foundation, West Lafayette, IN (US)

(21) Appl. No.: 14/206,904

(22) Filed: Mar. 12, 2014

Related U.S. Application Data
(60) Provisional application No. 61/787,747, filed on Mar. 15, 2013.

(51) Int. Cl. A61K 51/08; A61K 49/00

(52) U.S. Cl. CPC A61K 51/08; A61K 49/0052; A61K 49/0021
USPC 424/1.69; 424/9.6

(57) ABSTRACT

The invention relates to methods of imaging asthma using ligand conjugates. More particularly, the invention relates to the use of ligands that bind to cells associated with asthma conjugated to a chromophore, or to a chemical moiety capable of emitting radiation, for administration to a diseased host for imaging asthma.

### CROSS-REFERENCE TO RELATED APPLICATIONS

The present application claims, under 35 U.S.C. §119(e), the benefit of and priority to U.S. Provisional Application No. 61/787,747 filed Mar. 15, 2013, hereby incorporated by reference herein.

### FIELD OF THE INVENTION

This invention relates to a method for targeting cells associated with asthma using ligand conjugates. More particularly, ligands that bind to cells associated with asthma are conjugated to a chromophore, or to a chemical moiety capable of emitting radiation, for administration to a diseased host for imaging asthma.

### BACKGROUND AND SUMMARY OF THE INVENTION

Activated macrophages can participate in the immune response by nonspecifically engulfing and killing foreign pathogens within the macrophage, by displaying degraded peptides from foreign proteins on the macrophage cell surface where they can be recognized by other immune cells, and by secreting cytokines and other factors that modulate the function of T and B lymphocytes, resulting in further stimulation of immune responses. Activated macrophages can also contribute to the pathophysiology of disease in some instances.

Allergic asthma, the most common form of asthma, is a chronic inflammatory disease characterized by the development of a type 2 helper T cell (TH2) response towards inhaled allergens. Allergen-specific TH2 cells secrete cytokines that regulate the synthesis of allergen-specific immunoglobulin E, cause airway hyper-reactivity, stimulate mast cell infiltration, promote pulmonary eosinophilia, and induce accumulation of alternatively activated macrophages in the lungs. There is much scientific evidence for the prominent involvement of alternatively activated macrophages (AAMs) in allergic asthma.

The folate receptor-beta (FR-β), a homolog of the FR-α, is a glycosylphosphatidylinositol (GPI)-anchored membrane protein that is normally expressed in human placenta and on a subset of cells of myelomonocytic lineage. Although the FR-β has been detected on both CD34+ bone marrow cells and normal human neutrophils, the receptor on these cells is functionally inactive and unable to bind folate or folate-linked drugs. In contrast, a functional FR-β with nanomolar affinity for the vitamin has been identified on activated macrophages that accumulate in inflammatory diseases such as rheumatoid arthritis, atherosclerosis, systemic lupus erythrematosus, Crohn's disease, and osteoarthritis.

To determine whether ligand conjugates might be used in the targeting of agents to asthma, for example, targeting of imaging agents for the imaging and/or diagnosis of asthma, Applicants have isolated and characterized AAMs and have shown for the first time that AAMs express the FR-β and that this folate receptor is functionally active and useful for the selective delivery of folate-targeted agents to cells associated with asthma.

In accordance with Applicants' invention described herein, the embodiments of the following numbered clauses, or any combination thereof, are contemplated.

1. A method of imaging asthma, said method comprising the steps of:
administering to a patient afflicted with asthma an effective amount of a composition comprising a conjugate of the general formula

L-X

wherein the group L comprises a ligand, wherein the ligand is a folate, and wherein the group X comprises a chromophore capable of emitting light; and
imaging the asthma.

2. The method of clause 1 wherein the chromophore is selected from the group consisting of a fluorophore, a Raman enhancing dye, an hematoporphyrin, and derivatives thereof.

3. The method of clause 1 or 2 wherein the chromophore is a fluorophore.

4. The method of any one of clauses 1 to 3 wherein the chromophore is selected from the group consisting of a fluorescein, a rhodamine, a cyanine, a DyLight Fluor, and an Alexa Fluor.

5. The method of any one of clauses 1 to 4 wherein the chromophore has the formula

[chemical structure]

where X is oxygen, nitrogen, sulfur, S(O)₂, or C(O), and where X is attached via a divalent linker to the ligand; Y is OR^{a}, NR^{a}₂, or NR^{a}₃⁺; and Y′ is O, NR^{a}, or NR^{a}₂⁺; n is in each instance independently selected from 0, 1, 2, or 3; where each R is independently selected in each instance from H, alkyl, alkyloxy, heteroalkyl, fluoro, sulfonic acid, sulfonate, and salts thereof; and R^{a} is hydrogen, alkly, alkylsulfonic acid, or alkylsulfonate, and salts thereof; or at least one of R and Ra the atoms to which they are attached form a heterocycle.

6. The method of any one of clauses 1 to 4 wherein the chromophore has the formula

[chemical structure]

where X is oxygen, nitrogen, or sulfur, and where X is attached via a divalent linker to the ligand; and each R is independently selected in each instance from hydrogen, alkyl, heteroalkyl; and n is an integer from 0 to about 4.

7. The method of any one of clauses 1 to 4 wherein the chromophore has the formula

[chemical structure]

wherein R_{A} and R_{B} are independently selected in each instance from alkyl, heteroalkyl, alkylsulfonic acid, alkylsulfonate, or a salt thereof, or an amine or a derivative thereof; L₁ is an alkylene linked via a divalent linker to the ligand; R is independently selected in each instance from alkyl, heteroalkyl, or alkylsulfonic acid, or alkylsulfonate, or a salt thereof; n is independently in each instance an integer from 0 to about 3; x is an integer from about 1 to about 4; and Het is selected from the group consisting of

[chemical structure]

wherein * is the attachment point; and R^{C} is alkyl or heteroalkyl.

8. The method of any one of clauses 1 to 3 wherein the chromophore is selected from the group consisting of Cy3, Cy5, Cy7, Oregon Green 488, Oregon Green 514, AlexaFluor 488, AlexaFluor 647, tetramethylrhodamine, DyLight 680, CW 800, and Texas Red.

9. The method of any one of clauses 1 to 4 wherein the chromophore is fluorescein.

10. The method of any one of clauses 1 to 9 wherein the folate has the formula

[chemical structure]

wherein Y¹ and Y² are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;

A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R⁴, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

D is a divalent linker;

* represents the attachment point for X; and

n, p, r, s and t are each independently either 0 or 1.

11. The method of any one of clauses 1 to 9 wherein the folate has the formula

[chemical structure]

wherein * indicates the attachment point to a divalent linker attached to the chromophore.

12. A method of imaging asthma, said method comprising the steps of:

administering to a patient afflicted with asthma an effective amount of a composition comprising a conjugate of the general formula

L-X

wherein the group L comprises a ligand, wherein the ligand is a folate, and wherein the group X comprises a chemical moiety that emits radiation; and

imaging the asthma.

13. The method of clause 12 wherein the group X comprises a metal chelating moiety that chelates a metal cation.

14. The method of clause 13 wherein the metal cation is a radionuclide.

15. The method of clause 14 wherein the radionuclide is ^{99m}Tc.

16. The method of clause 13 wherein the metal cation is a nuclear magnetic resonance imaging enhancing agent.

17. The method of any one of clauses 12 to 16 wherein the folate has the formula

[chemical structure]

wherein Y¹ and Y² are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;

A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

D is a divalent linker;

* represents the attachment point for X; and

n, p, r, s and t are each independently either 0 or 1.

18. The method of any one of clauses 12 to 15 wherein the conjugate comprises a compound of the formula

[chemical structure]

wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein the compound is bound to a radionuclide.

19. The method of any one of clauses 12 to 15 or 18 wherein the conjugate has the formula

[chemical structure]

wherein the conjugate is bound to an isotope of technicium and wherein the isotope of technicium is ^{99m}Tc.

20. The method of any one of clauses 12 to 15 or 18 to 19 wherein the folate has the formula

[chemical structure]

wherein * indicates the attachment point to a divalent linker attached to the group X.

21. A method of targeting a ligand conjugate to cells associated with asthma, said method comprising the steps of:
administering to a patient afflicted with asthma a composition comprising a conjugate of the general formula

L-X

wherein the group L comprises a ligand and wherein the ligand is a folate.

22. The method of clause 21 wherein the cells associated with asthma are alternatively activated macrophages.

23. The method of clause 21 or 22 wherein the group X is a chromophore.

24. The method of clause 23 wherein the chromophore is selected from the group consisting of a fluorophore, a Raman enhancing dye, an hematoporphyrin, and derivatives thereof.

25. The method of clause 23 or 24 wherein the chromophore is a fluorophore.

26. The method of clause 23 or 24 wherein the chromophore is selected from the group consisting of a fluorescein, a rhodamine, a cyanine, a DyLight Fluor, and an Alexa Fluor.

27. The method of any one of clauses 23 to 25 wherein the chromophore has the formula

[chemical structure]

where X is oxygen, nitrogen, sulfur, S(O)₂, or C(O), and where X is attached via a divalent linker to the ligand; Y is OR^{a}, NR^{a}₂, or NR^{a}₃⁺; and Y′ is O, NR^{a}, or NR^{a}₂⁺; n is in each instance independently selected from 0, 1, 2, or 3; where each R is independently selected in each instance from H, alkyl, alkyloxy, heteroalkyl, fluoro, sulfonic acid, sulfonate, and salts thereof; and R^{a} is hydrogen, alkly, alkylsulfonic acid, or alkylsulfonate, and salts thereof; or at least one of R and Ra the atoms to which they are attached form a heterocycle.

28. The method of any one of clauses 23 to 25 wherein the chromophore has the formula

[chemical structure]

where X is oxygen, nitrogen, or sulfur, and where X is attached via a divalent linker to the ligand; and each R is independently selected in each instance from hydrogen, alkyl, heteroalkyl; and n is an integer from 0 to about 4.

29. The method of any one of clauses 23 to 25 wherein the chromophore has the formula

[chemical structure]

wherein R_{A} and R_{B} are independently selected in each instance from alkyl, heteroalkyl, alkylsulfonic acid, alkylsulfonate, or a salt thereof, or an amine or a derivative thereof; L₁ is an alkylene linked via a divalent linker to the ligand; R is independently selected in each instance from alkyl, heteroalkyl, or alkylsulfonic acid, or alkylsulfonate, or a salt thereof; n is independently in each instance an integer from 0 to about 3; x is an integer from about 1 to about 4; and Het is selected from the group consisting of

[chemical structure]

wherein * is the attachment point; and R^{C} is alkyl or heteroalkyl.

30. The method of any one of clauses 23 to 25 wherein the chromophore is selected from the group consisting of Cy3, Cy5, Cy7, Oregon Green 488, Oregon Green 514, AlexaFluor 488, AlexaFluor 647, tetramethylrhodamine, DyLight 680, CW 800, and Texas Red.

31. The method of any one of clauses 23 to 25 wherein the chromophore is fluorescein.

32. The method of clause 21 wherein the group X comprises a metal chelating moiety that chelates a metal cation.

33. The method of clause 32 wherein the metal cation is a radionuclide.

34. The method of clause 33 wherein the radionuclide is ^{99m}Tc.

35. The method of clause 32 wherein the metal cation is a nuclear magnetic resonance imaging enhancing agent.

36. The method of any one of clauses 21 to 35 wherein the folate has the formula

[chemical structure]

wherein Y¹ and Y² are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;

A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

D is a divalent linker;

* represents the attachment point for X; and

n, p, r, s and t are each independently either 0 or 1.

37. The method of any one of clauses 21 or 32 to 35 wherein the conjugate comprises a compound of the formula

[chemical structure]

wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein the compound is bound to a radionuclide.

38. The method of any one of clauses 21, 32 to 35, or 37 wherein the conjugate has the formula

[chemical structure]

wherein the conjugate is bound to an isotope of technicium and wherein the isotope of technicium is ^{99m}Tc.

39. The method of any one of clauses 21 to 35 wherein the folate has the formula

[chemical structure]

wherein * indicates the attachment point to a divalent linker attached to the group X.

### BRIEF DESCRIPTION OF THE DRAWINGS

FIG. 1 shows analysis of FR-β transcripts in murine lung tissue during OVA-induced acute allergic inflammation related to asthma. Balb/c mice (n=4) were sensitized and challenged with OVA to induce experimental asthma or treated similarly with saline (n=4) and used as controls. Panel A shows representative photographs of H&E (×100) stained lung tissue sections from control (PBS/PBS) and OVA-induced (OVA/OVA) asthmatic mice. Panel B shows measurement of arginase activity in the lungs of control (PBS/PBS) and OVA-induced (OVA/OVA) asthmatic mice (P<0.05). Panel C shows expression of FR-β or hypoxanthine phosphoribosyltransferase (HPRT; control) transcripts in lung tissues from control (PBS/PBS) and OVA-induced (OVA/OVA) asthmatic mice as detected by RT-PCR.

FIG. 2 shows FR-β expression on F4/80⁺ macrophages in lungs from mice during OVA-induced acute allergic inflammation related to asthma. Lungs from asthmatic and control mice were excised, sliced into small cubes and processed for preparation of single cell suspensions. Panel A shows the percentage of F4/80⁺ or CD68⁺ macrophages within the FR⁺ cell population analyzed by flow cytometry after staining with rabbit anti-FR primary antibody followed by fluorescein isothiocyanate (FITC)-conjugated anti-rabbit IgG secondary antibody to define the FR⁺ cell population. Panel B shows the expression of various FR isoforms (FR-α, FR-β, and FR-δ) in FR⁺F4/80⁺ cells sorted and analyzed by real-time PCR. Panel C shows the total RNA isolated from sorted F4/80⁺ macrophages, converted to cDNA, and analyzed using PCR analysis of FR-β or HPRT (control).

FIG. 3 shows an increase in F4/80⁺ cells expressing FR-β in lung tissue during OVA-induced asthma in mice. Mononuclear cells isolated from lungs of mice during OVA-induced experimental asthma were analyzed by flow cytometry for F4/80 expression (a macrophage marker) and for FR-β expression. Panel A shows the percentage of FR⁺ cells within the F4/80⁺ macrophage population. Panel B shows mice (n=6) with OVA-induced (OVA/OVA) airway inflammation have significantly more FR⁺F4/80⁺ cells in their lung tissue than control (PBS/PBS) mice (n=6). Pairwise comparison using t test with pooled SD were used for statistical analyses (P<0.0001).

FIG. 4 shows FR-β expressed on asthmatic lung macrophages can bind folate-conjugates. Panel A shows macrophages isolated from lungs of mice with OVA-induced (OVA/OVA) experimental asthma (gated by side scatter and F4/80⁺ fluorescence) were examined for binding of FOG. Flow cytometry showed strong surface binding of FOG after 1 hour of incubation. Moreover, binding of FOG could be completely blocked by 1000-fold excess of free folic acid (see filled histogram). Panel B shows ex vivo near-infrared fluorescent (NIRF) images of representative lungs from control (PBS/PBS; left panel) and asthmatic mice (OVA/OVA); middle and right panels) following intravenous (i.v.) injection of folate-Daylight 680 in the absence (left and middle panels) or presence (right panel) of 100-fold molar excess of free folate-glucosamine to block all FR-β.

FIG. 5 shows FR⁺F4/80⁺ cells express markers consistent with an alternatively activated macrophage phenotype. Panels A and B show expression of FR-β and either iNOS (Panel A) or mannose receptor (Panel B) in macrophages harvested from lungs of OVA-induced (OVA/OVA) mice or control (PBS/PBS) mice. Panel C shows quantitative PCR results demonstrating expression of iNOS and mannose receptor mRNAs (relative to 18S rRNA). Total RNA was prepared from sorted FR⁺F4/80⁺ cells collected from lungs of OVA-induced mice (n=3). Panel D shows arginase activity in the sorted FR⁺ macrophages from lungs of OVA-induced (FR-β+ Macrophages) mice or in the sorted naïve macrophages from lungs of control mice (Naive Macrophages) (n=3 mice in each group).

FIG. 6 shows that folate-linked radioimaging agents can be used to assess the activation status of macrophages in asthmatic lungs in vivo. Panel A shows representative standard γ-scintigraphic images demonstrating ^{99m}Tc-EC20 uptake in the lungs of control mice (PBS/PBS; left), OVA-induced mice (OVA/OVA; middle), or OVA-induced mice plus 100-fold excess free folic acid (Competition; far right). Radioimages were obtained on Kodak Imaging Station 4 hours after injection. Panel B shows representative SPECT/CT images of control mice (PBS/PBS; left), OVA-induced mice (OVA/OVA; middle), or OVA-induced mice plus 100-fold excess free folic acid (Competition; far right). SPECT/CT scans were performed 2 hours after injection. Panel C shows ex vivo γ-scintigraphic images of lungs excised from euthanized mice from control mice (PBS/PBS; far right), OVA-induced mice (OVA/OVA; left), or OVA-induced mice plus 100-fold excess free folic acid (Competition; middle). Panel D shows uptake of ^{99m}Tc-EC20 in lungs from control mice (PBS/PBS; left), OVA-induced mice (OVA/OVA; middle), or OVA-induced mice plus 100-fold excess free folic acid (Competition; far right). (n=6 mice in each group). Lungs from different groups were harvested, and the uptake of ^{99m}Tc-EC20 was determined by γ-counting. ***P<0.0001.

### DETAILED DESCRIPTION OF THE INVENTION

In accordance with Applicants' invention described herein, the embodiments of the following numbered clauses, or any combination thereof, are contemplated.

1. A method of imaging asthma, said method comprising the steps of:
administering to a patient afflicted with asthma an effective amount of a composition comprising a conjugate of the general formula

L-X

wherein the group L comprises a ligand, wherein the ligand is a folate, and wherein the group X comprises a chromophore capable of emitting light; and
imaging the asthma.

2. The method of clause 1 wherein the chromophore is selected from the group consisting of a fluorophore, a Raman enhancing dye, an hematoporphyrin, and derivatives thereof.

3. The method of clause 1 or 2 wherein the chromophore is a fluorophore.

4. The method of any one of clauses 1 to 3 wherein the chromophore is selected from the group consisting of a fluorescein, a rhodamine, a cyanine, a DyLight Fluor, and an Alexa Fluor.

5. The method of any one of clauses 1 to 4 wherein the chromophore has the formula

[chemical structure]

where X is oxygen, nitrogen, sulfur, S(O)₂, or C(O), and where X is attached via a divalent linker to the ligand; Y is OR^{a}, NR^{a}₂, or NR^{a}₃⁺; and Y′ is O, NR^{a}, or NR^{a}₂⁺; n is in each instance independently selected from 0, 1, 2, or 3; where each R is independently selected in each instance from H, alkyl, alkyloxy, heteroalkyl, fluoro, sulfonic acid, sulfonate, and salts thereof; and R^{a} is hydrogen, alkly, alkylsulfonic acid, or alkylsulfonate, and salts thereof; or at least one of R and Ra the atoms to which they are attached form a heterocycle.

6. The method of any one of clauses 1 to 4 wherein the chromophore has the formula

[chemical structure]

where X is oxygen, nitrogen, or sulfur, and where X is attached via a divalent linker to the ligand; and each R is independently selected in each instance from hydrogen, alkyl, heteroalkyl; and n is an integer from 0 to about 4.

7. The method of any one of clauses 1 to 4 wherein the chromophore has the formula

[chemical structure]

wherein R_{A} and R_{B} are independently selected in each instance from alkyl, heteroalkyl, alkylsulfonic acid, alkylsulfonate, or a salt thereof, or an amine or a derivative thereof; L₁ is an alkylene linked via a divalent linker to the ligand; R is independently selected in each instance from alkyl, heteroalkyl, or alkylsulfonic acid, or alkylsulfonate, or a salt thereof; n is independently in each instance an integer from 0 to about 3; x is an integer from about 1 to about 4; and Het is selected from the group consisting of

[chemical structure]

wherein * is the attachment point; and R_{C} is alkyl or heteroalkyl.

8. The method of any one of clauses 1 to 3 wherein the chromophore is selected from the group consisting of Cy3, Cy5, Cy7, Oregon Green 488, Oregon Green 514, AlexaFluor 488, AlexaFluor 647, tetramethylrhodamine, DyLight 680, CW 800, and Texas Red.

9. The method of any one of clauses 1 to 4 wherein the chromophore is fluorescein.

10. The method of any one of clauses 1 to 9 wherein the folate has the formula

[chemical structure]

wherein Y¹ and Y² are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C≡C—;

A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

D is a divalent linker;

* represents the attachment point for X; and

n, p, r, s and t are each independently either 0 or 1.

11. The method of any one of clauses 1 to 9 wherein the folate has the formula

[chemical structure]

wherein * indicates the attachment point to a divalent linker attached to the chromophore.

12. A method of imaging asthma, said method comprising the steps of:
administering to a patient afflicted with asthma an effective amount of a composition comprising a conjugate of the general formula

L-X

wherein the group L comprises a ligand, wherein the ligand is a folate, and wherein the group X comprises a chemical moiety that emits radiation; and
imaging the asthma.

13. The method of clause 12 wherein the group X comprises a metal chelating moiety that chelates a metal cation.

14. The method of clause 13 wherein the metal cation is a radionuclide.

15. The method of clause 14 wherein the radionuclide is ^{99m}Tc.

16. The method of clause 13 wherein the metal cation is a nuclear magnetic resonance imaging enhancing agent.

17. The method of any one of clauses 12 to 16 wherein the folate has the formula

[chemical structure]

wherein Y¹ and Y² are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;

A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

D is a divalent linker;

* represents the attachment point for X; and

n, p, r, s and t are each independently either 0 or 1.

18. The method of any one of clauses 12 to 15 wherein the conjugate comprises a compound of the formula

[chemical structure]

wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein the compound is bound to a radionuclide.

19. The method of any one of clauses 12 to 15 or 18 wherein the conjugate has the formula

[chemical structure]

wherein the conjugate is bound to an isotope of technicium and wherein the isotope of technicium is ^{99m}Tc.

20. The method of any one of clauses 12 to 15 or 18 to 19 wherein the folate has the formula

[chemical structure]

wherein * indicates the attachment point to a divalent linker attached to the group X.

21. A method of targeting a ligand conjugate to cells associated with asthma, said method comprising the steps of:
administering to a patient afflicted with asthma a composition comprising a conjugate of the general formula

L-X

wherein the group L comprises a ligand and wherein the ligand is a folate.

22. The method of clause 21 wherein the cells associated with asthma are alternatively activated macrophages.

23. The method of clause 21 or 22 wherein the group X is a chromophore.

24. The method of clause 23 wherein the chromophore is selected from the group consisting of a fluorophore, a Raman enhancing dye, an hematoporphyrin, and derivatives thereof.

25. The method of clause 23 or 24 wherein the chromophore is a fluorophore.

26. The method of clause 23 or 24 wherein the chromophore is selected from the group consisting of a fluorescein, a rhodamine, a cyanine, a DyLight Fluor, and an Alexa Fluor.

27. The method of any one of clauses 23 to 25 wherein the chromophore has the formula

[chemical structure]

where X is oxygen, nitrogen, sulfur, S(O)₂, or C(O), and where X is attached via a divalent linker to the ligand; Y is OR^{a}, NR^{a}₂, or NR^{a}₃⁺; and Y′ is O, NR^{a}, or NR^{a}₂⁺; n is in each instance independently selected from 0, 1, 2, or 3; where each R is independently selected in each instance from H, alkyl, alkyloxy, heteroalkyl, fluoro, sulfonic acid, sulfonate, and salts thereof; and R^{a} is hydrogen, alkly, alkylsulfonic acid, or alkylsulfonate, and salts thereof; or at least one of R and Ra the atoms to which they are attached form a heterocycle.

28. The method of any one of clauses 23 to 25 wherein the chromophore has the formula

[chemical structure]

where X is oxygen, nitrogen, or sulfur, and where X is attached via a divalent linker to the ligand; and each R is independently selected in each instance from hydrogen, alkyl, heteroalkyl; and n is an integer from 0 to about 4.

29. The method of any one of clauses 23 to 25 wherein the chromophore has the formula

[chemical structure]

wherein R_{A} and R_{B} are independently selected in each instance from alkyl, heteroalkyl, alkylsulfonic acid, alkylsulfonate, or a salt thereof, or an amine or a derivative thereof; L₁ is an alkylene linked via a divalent linker to the ligand; R is independently selected in each instance from alkyl, heteroalkyl, or alkylsulfonic acid, or alkylsulfonate, or a salt thereof; n is independently in each instance an integer from 0 to about 3; x is an integer from about 1 to about 4; and Het is selected from the group consisting of

[chemical structure]

wherein * is the attachment point; and R_{C} is alkyl or heteroalkyl.

30. The method of any one of clauses 23 to 25 wherein the chromophore is selected from the group consisting of Cy3, Cy5, Cy7, Oregon Green 488, Oregon Green 514, AlexaFluor 488, AlexaFluor 647, tetramethylrhodamine, DyLight 680, CW 800, and Texas Red.

31. The method of any one of clauses 23 to 25 wherein the chromophore is fluorescein.

32. The method of clause 21 wherein the group X comprises a metal chelating moiety that chelates a metal cation.

33. The method of clause 32 wherein the metal cation is a radionuclide.

34. The method of clause 33 wherein the radionuclide is ^{99m}Tc.

35. The method of clause 32 wherein the metal cation is a nuclear magnetic resonance imaging enhancing agent.

36. The method of any one of clauses 21 to 35 wherein the folate has the formula

[chemical structure]

wherein Y¹ and Y² are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;

A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

D is a divalent linker;

* represents the attachment point for X; and

n, p, r, s and t are each independently either 0 or 1.

37. The method of any one of clauses 21 or 32 to 35 wherein the conjugate comprises a compound of the formula

[chemical structure]

wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein the compound is bound to a radionuclide.

38. The method of any one of clauses 21, 32 to 35, or 37 wherein the conjugate has the formula

[chemical structure]

wherein the conjugate is bound to an isotope of technicium and wherein the isotope of technicium is ^{99m}Tc.

39. The method of any one of clauses 21 to 35 wherein the folate has the formula

[chemical structure]

wherein * indicates the attachment point to a divalent linker attached to the group X.

As used herein, the phrase “ligand conjugate” is equivalent to the conjugate of general formula L-X.

As used herein, the phrase “chemical moiety that emits radiation” refers to a chemical moiety that directly or indirectly emits radiation. An exemplary embodiment of a chemical moiety that indirectly emits radiation is a metal chelator that binds a metal. In this embodiment, the metal can be a metal cation. In another embodiment, the metal cation can be a radionuclide. Thus, in this embodiment, the chemical moiety that emits radiation comprises a chelator that does not directly emit radiation, bound to a metal cation (e.g., a radionuclide) that directly emits radiation.

As used herein, the word “imaging” or “detecting” refers to identifying by imaging the presence of ligand conjugates (e.g., folate conjugates) associated with asthma in the lungs of a patient afflicted with asthma.

In one embodiment, the ligand conjugates (e.g., folate conjugates) bind to alternatively activated macrophages associated with asthma. The ligand conjugates may be associated with alternatively activated macrophages, e.g., having arginase-1 (Arg1) or acidic mammalian chitinase (AMCase) markers, for example, in the patient afflicted with asthma. In one illustrative aspect, the ligand conjugates are capable of preferentially binding to alternatively activated macrophages compared to resting macrophages in the lung due to preferential expression of the receptor for the ligand on the alternatively activated macrophages.

In another illustrative aspect, the light or radiation emitted by the ligand-chromophore conjugate or the ligand-chemical moiety conjugate, respectively, can be detected externally using such methods as X-ray detection.

In one embodiment, the ligand (L) is folate, a folate analog or derivative, or another folate receptor binding molecule.

In other embodiments, the ligand (L) is a folate. It is to be understood as used herein, that the term a folate is used both individually and collectively to refer to folate itself, and to such analogs and derivatives of folate as are capable of binding to folate receptors.

Illustrative embodiments of folate analogs and/or derivatives include folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refer to the art-recognized analogs having a carbon atom substituted for one or two nitrogen atoms in the naturally occurring folic acid structure, or analog or derivative thereof. For example, the deaza analogs include the 1-deaza, 3-deaza, 5-deaza, 8-deaza, and 10-deaza analogs of folate. The dideaza analogs include, for example, 1,5-dideaza, 5,10-dideaza, 8,10-dideaza, and 5,8-dideaza analogs of folate. Other folates useful as complex forming ligands include the folate receptor-binding analogs aminopterin, amethopterin (methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate). The foregoing folic acid analogs and/or derivatives are included within the term a folate, reflecting their ability to bind to folate receptors.

Additional analogs of folate that bind to folate receptors and that are included within the term a folate are described in US Patent Application Publication Serial Nos. 2005/0227985 and 2004/0242582, the disclosures of which are incorporated herein by reference. Illustratively, such folate analogs have the general formula:

[chemical structure]

wherein Y¹ and Y² are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C≡C—;

A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

D is a divalent linker;

* represents the attachment point for X; and

n, p, r, s and t are each independently either 0 or 1.

In another embodiment, the alternatively activated macrophage binding ligand is a specific monoclonal or polyclonal antibody or Fab or scFv (i.e., a single chain variable region) fragment of an antibody capable of preferential binding to alternatively activated macrophages as compared to resting macrophages.

In various embodiments, the binding site for the ligand L can include receptors for any ligand molecule, or a derivative or analog thereof, capable of preferentially binding to a receptor uniquely expressed or preferentially expressed/presented on the surface of alternatively activated macrophages associated with asthma. For example, a surface-presented protein uniquely expressed or preferentially expressed by alternatively activated macrophages associated with asthma is a receptor that is either not present or is present at insignificant concentrations on resting macrophages providing a means for preferential detection of alternatively activated macrophages associated with asthma.

Accordingly, any receptor that is upregulated on alternatively activated macrophages, associated with asthma, compared to resting macrophages, or which is not expressed/presented on the surface of resting macrophages, or any receptor that is not expressed/presented on the surface of resting macrophages in significant amounts could be used for targeting. In one embodiment, the site that binds the ligand conjugates used in accordance with the present invention is a vitamin receptor, for example, the folate receptor, which binds folate, or an analog or derivative thereof.

In accordance with the invention the ligand conjugates can bind with high affinity to receptors on alternatively activated macrophages associated with asthma. In one embodiment, the high affinity binding can be inherent to the ligand or the binding affinity can be enhanced by the use of a chemically modified ligand (e.g., an analog or a derivative) or by the particular chemical linkage, in the ligand conjugate, between the ligand and the chromophore or between the ligand and the chemical moiety that emits radiation (e.g., a chemical moiety capable of emitting radiation).

In one illustrative aspect, the chemical linkage in the ligand conjugate between the ligand and the chromophore or between the ligand and the chemical moiety that emits radiation can be a direct linkage or the linkage can be through an intermediary linker. In one embodiment, if present, an intermediary linker can be any biocompatible linker known in the art. In one illustrative embodiment, the linker comprises about 1 to about 30 carbon atoms. In another illustrative embodiment, the linker comprises about 2 to about 20 carbon atoms. In other embodiments, lower molecular weight linkers (i.e., those having an approximate molecular weight of about 30 to about 300) are employed.

In one embodiment, the linker comprises a heteroatom directly bonded to the ligand and the chromophore or to the ligand and the chemical moiety that emits radiation. In one embodiment, the heteroatom is nitrogen. In another embodiment, the linker comprises an optionally-substituted diaminoalkylene. In one embodiment, the optionally-substituted diaminoalkylene is a diaminoacid. In another embodiment, the linker comprises one or more optionally-substituted diaminoalkylene moieties, and one or more optionally-substituted amino acids. In one illustrative example, the linker comprises glutamic acid.

In another illustrative embodiment, the linker includes one or more amino acids. In one variation, the linker includes a single amino acid. In another variation, the linker includes a peptide having from 2 to about 50, 2 to about 30, or 2 to about 20 amino acids. In another variation, the linker includes a peptide having from about 4 to about 8 amino acids. Such amino acids are illustratively selected from the naturally occurring amino acids, or stereoisomers thereof. In another embodiment, the amino acid may also be any other amino acid, such as any amino acid having the general formula:

—N(R)—(CR′R″)_{q}—C(O)—

where R is hydrogen, alkyl, acyl, or a suitable nitrogen protecting group, R′ and R″ are hydrogen or a substituent, each of which is independently selected in each occurrence, and q is an integer such as 1, 2, 3, 4, or 5. Illustratively, R′ and/or R″ independently correspond to, but are not limited to, hydrogen or the side chains present on naturally occurring amino acids, such as methyl, benzyl, hydroxymethyl, thiomethyl, carboxyl, carboxylmethyl, guanidinopropyl, and the like, and derivatives and protected derivatives thereof. The above described formula includes all stereoisomeric variations. For example, the amino acid may be selected from asparagine, aspartic acid, cysteine, glutamic acid, lysine, glutamine, arginine, serine, ornithine, threonine, and the like. In one variation, the linker includes at least 2 amino acids selected from asparagine, aspartic acid, cysteine, glutamic acid, lysine, glutamine, arginine, serine, ornithine, and threonine. In another variation, the linker includes between 2 and about 5 amino acids selected from asparagine, aspartic acid, cysteine, glutamic acid, lysine, glutamine, arginine, serine, ornithine, and threonine. In another variation, the linker includes a tripeptide, tetrapeptide, pentapeptide, or hexapeptide consisting of amino acids selected from aspartic acid, cysteine, glutamic acid, lysine, arginine, and ornithine, and combinations thereof.

In another embodiment, the linker may also include one or more spacer linkers. Illustrative spacer linkers are shown in the following table

The following non-limiting, illustrative spacer linkers are described where * indicates the point of attachment.

[chemical structure]

[chemical structure]

Generally, any manner of forming a complex between the ligand and the chromophore, between the ligand and the chemical moiety that emits radiation, between a linker and the ligand, or between a linker and the chromophore or chemical moiety that emits radiation can be utilized in accordance with the present invention. With or without a linker, the complex can be formed by conjugation of the components of the ligand conjugate, for example, through hydrogen, ionic, or covalent bonds. Covalent bonding of the components of the conjugate can occur, for example, through the formation of amide, ester, disulfide, or imino bonds between acid, aldehyde, hydroxy, amino, sulfhydryl, or hydrazo groups. Also, in some embodiments, a linker can comprise an indirect means for associating the ligand with the chromophore/chemical moiety that emits radiation, such as by connection through spacer arms or bridging molecules. The direct or indirect means for association should not prevent the binding of the ligand to the receptor on the alternatively activated macrophages for operation of the method of the present invention. Alternatively, the ligand conjugate can be one comprising a liposome wherein the chemical moiety that emits radiation, for example, is contained within a liposome which is itself covalently linked to the alternatively activated macrophage-binding ligand.

In the embodiment where the ligand is folate, an analog/derivative of folate, or any other folate receptor binding molecule, folate can be conjugated to the chromophore or chemical moiety that emits radiation by an art-recognized procedure that utilizes trifluoroacetic anhydride to prepare γ-esters of folic acid via a pteroyl azide intermediate. This procedure results in the synthesis of a folate ligand, conjugated to the chromophore or chemical moiety that emits radiation only through the γ-carboxy group of the glutamic acid groups of folate.

Alternatively, folic acid can be coupled by art-recognized procedures through the α-carboxy moiety of the glutamic acid group, or through both the α and γ carboxylic acid entities.

In various illustrative embodiments, the amount of the ligand conjugate effective for use in accordance with the method of the invention depends on many parameters, including the molecular weight of the ligand conjugate, its route of administration, and its tissue distribution. In one embodiment, an “effective amount” of the ligand conjugate is an amount sufficient to be useful in imaging for the detection of asthma in a patient afflicted with asthma, or for targeting the ligand conjugate to the site of the asthma. In another embodiment, the effective amount of the ligand conjugate to be administered to a patient afflicted with asthma can range from about 1 ng/kg to about 10 mg/kg, or from about 10 g/kg to about 1 mg/kg, or from about 100 μg/kg to about 500 μg/kg.

In one illustrative aspect, the ligand conjugate can be administered in one or more doses (e.g., about 1 to about 3 doses) prior to, for example, an external imaging procedure. The number of doses depends on the molecular weight of the ligand conjugate, its route of administration, and its tissue distribution, among other factors. In the embodiment where external imaging is used, the external imaging procedure is typically performed about 0.1 to about 6 hours post-administration of the ligand conjugate to the patient afflicted with asthma, but the external imaging procedure can be performed at any time post-administration of the ligand conjugate as long as the asthma (e.g., cells associated with asthma) is detectable.

In one illustrative embodiment, the ligand conjugates are administered parenterally to the patient afflicted with asthma, for example, intravenously, intradermally, subcutaneously, intramuscularly, or intraperitoneally, in combination with a pharmaceutically acceptable carrier. Suitable means for parenteral administration include needle (including microneedle) injectors, needle-free injectors and infusion techniques. Alternatively, the conjugates can be administered to the patient afflicted with asthma by other medically useful procedures such as in an orally available formulation. In accordance with the invention, a “patient afflicted with asthma” means any patient suspected of having asthma, whether symptomatic or not, and whether that patient is actually diagnosed with asthma or not, who would benefit from an imaging procedure using the method of the present invention.

Examples of parenteral dosage forms include aqueous solutions of the ligand conjugate, for example, a solution in isotonic saline, 5% glucose or other well-known pharmaceutically acceptable liquid carriers such as alcohols, glycols, esters and amides. In one embodiment, the parenteral compositions for use in accordance with this invention can be in the form of a reconstitutable lyophilizate comprising the one or more doses of the ligand conjugate.

In other embodiments, the ligand conjugates can be in the form of a composition. In other embodiments, pharmaceutically acceptable salts of the ligand conjugates described herein can be used. Pharmaceutically acceptable salts of the ligand conjugates described herein include the acid addition and base salts thereof.

Suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts of the ligand conjugates described herein are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

In one embodiment, the ligand conjugates described herein may be administered as a composition in association with one or more pharmaceutically acceptable carriers. In one embodiment, the carriers can be excipients. The choice of carrier will to a large extent depend on factors such as the particular mode of administration, the effect of the carrier on solubility and stability, and the nature of the dosage form. Pharmaceutical compositions suitable for the delivery of the ligand conjugates described herein and methods for their preparation will be readily apparent to those skilled in the art. In some embodiments, such compositions and methods for their preparation may be found, for example, in Remington: The Science & Practice of Pharmacy, 21th Edition (Lippincott Williams & Wilkins, 2005), incorporated herein by reference.

In some illustrative embodiments, formulations of the ligand conjugates for use in the methods described herein for parenteral administration comprise: a) a pharmaceutically active amount of the ligand conjugate; b) a pharmaceutically acceptable pH buffering agent to provide a pH in the range of about pH 4.5 to about pH 9; c) an ionic strength modifying agent in the concentration range of about 0 to about 250 millimolar; or d) a water soluble viscosity modifying agent in the concentration range of about 0.5% to about 7% total formula weight; or any combinations of a), b), c) and d).

In various illustrative embodiments, the pH buffering agents for use in compositions containing the ligand conjugates for use in the methods described herein are those agents known to the skilled artisan and include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, and MES.

In another illustrative embodiment, the ionic strength modulating agents include agents known in the art, for example, glycerin, propylene glycol, mannitol, glucose, dextrose, sorbitol, sodium chloride, potassium chloride, and other electrolytes.

In illustrative aspects, useful viscosity modulating agents include but are not limited to, ionic and non-ionic water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, chitosans, gellans or any combination thereof. In one embodiment, non-acidic viscosity enhancing agents, such as a neutral or basic agent can be employed in order to facilitate achieving the desired pH of the formulation. If a uniform gel is desired, dispersing agents such as alcohol, sorbitol or glycerin can be added, or the gelling agent can be dispersed by trituration, mechanical mixing, or stirring, or combinations thereof. In one embodiment, the viscosity enhancing agent can also provide the base, discussed above.

In one illustrative aspect, a pharmaceutically acceptable carrier can be a solvent, dispersion media, coatings, antibacterial and antifungal agents, isotonic and absorption delaying agents, and the like, and combinations thereof, that are physiologically compatible.

In some embodiments, the carrier is suitable for parenteral administration. Pharmaceutically acceptable carriers include sterile aqueous solutions or dispersions and sterile powders for the extemporaneous preparation of sterile injectable solutions or dispersions.

In various embodiments, liquid formulations may include suspensions and solutions. Such formulations may comprise a carrier, for example, water, ethanol, polyethylene glycol, propylene glycol, methylcellulose or a suitable oil, and one or more emulsifying agents and/or suspending agents.

In one embodiment, an aqueous suspension may contain the active materials (e.g., imaging agents) in admixture with appropriate excipients. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions may also contain one or more preservatives, for example, ascorbic acid, ethyl, n-propyl, or p-hydroxybenzoate; or one or more coloring agents.

Suitable emulsifying agents may be naturally-occurring gums, for example, gum acacia or gum tragacanth; naturally-occurring phosphatides, for example, soybean lecithin; and esters including partial esters derived from fatty acids and hexitol anhydrides, for example, sorbitan mono-oleate, and condensation products of the said partial esters with ethylene oxide, for example, polyoxyethylene sorbitan monooleate.

In other embodiments, isotonic agents, for example, sugars, polyalcohols such as mannitol, sorbitol, or sodium chloride can be included in the composition for use in the method of the invention.

In one aspect, a ligand conjugate as described herein may be administered directly into the blood stream, into muscle, or into an internal organ. Suitable routes for such parenteral administration include intravenous, intraarterial, intraperitoneal, intramuscular and subcutaneous delivery. Suitable means for parenteral administration include needle (including microneedle) injectors, needle-free injectors and infusion techniques.

In one illustrative aspect, parenteral formulations can be aqueous solutions, but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization under sterile conditions, may readily be accomplished using standard pharmaceutical techniques well known to those skilled in the art. In one embodiment, the solubility of a ligand conjugate used in the preparation of a parenteral formulation may be increased by the use of appropriate formulation techniques, such as the incorporation of solubility-enhancing agents.

In one embodiment, sterile injectable solutions can be prepared by incorporating the active agent (e.g., imaging agent) in the required amount in an appropriate solvent with one or a combination of ingredients described above, as required, followed by filtered sterilization. Typically, dispersions are prepared by incorporating the active compound into a sterile vehicle which contains a dispersion medium and any additional ingredients from those described above. In the case of sterile powders for the preparation of sterile injectable solutions, the preferred methods of preparation are vacuum drying and freeze-drying which yields a powder of the active ingredient (e.g., imaging agent) plus any additional desired ingredient from a previously sterile-filtered solution thereof.

In one illustrative aspect, the ligand conjugates are administered as an imaging composition comprising a ligand conjugate and a pharmaceutically acceptable carrier. In this embodiment, the ligand conjugate is formulated for parenteral administration and is administered to the patient in an amount effective to enable imaging of asthma. In this illustrative aspect, the nature of the chromophore or chemical moiety that emits radiation is dictated by the methodology used for external imaging, for example. Thus, for example, the chromophore can comprise a fluorophore, such as fluorescein, (see PCT publication number WO 01/074382, incorporated herein by reference, for a description of a ligand-fluorophore conjugate) or another chromophore such as rhodamine, coumarin, cyanine, HiLyte Fluors, DyLight Fluors, or Alexa Fluors, Texas Red, phycoerythrin, Oregon Green, Cy3, Cy5, Cy7, and the like, an hematoporphyrin, or a derivative thereof, or a Raman enhancing dye or agent, or a long wavelength fluorescent dye with optical properties that allow imaging through many layers of tissue. In another embodiment, the component of the ligand conjugate used for imaging can be a chemical moiety that emits radiation, such as a chelating moiety bound to a metal cation, for example, a radionuclide.

In another aspect, the chromophore can be a fluorescent agent selected from Oregon Green fluorescent agents, including but not limited to Oregon Green 488, Oregon Green 514, and the like, AlexaFluor fluorescent agents, including but not limited to AlexaFluor 488, AlexaFluor 647, and the like, fluorescein, and related analogs, rhodamine fluorescent agents, including but not limited to tetramethylrhodamine, and the like, DyLight fluorescent agents, including but not limited to DyLight 680, and the like, CW 800, Texas Red, phycoerythrin, and others. Illustrative fluorescent agents (e.g., chromophores) are shown in the following illustrative general structures:

[chemical structure]

where X is oxygen, nitrogen, sulfur, S(O)₂, or C(O), and where X is attached to linker L; Y is OR^{a}, NR^{a}₂, or NR^{a}₃⁺; and Y′ is O, NR^{a}, or NR^{a}₂⁺; n is in each instance independently selected from 0, 1, 2, or 3; where each R is independently selected in each instance from H, alkyl, alkyloxy, heteroalkyl, fluoro, sulfonic acid, sulfonate, and salts thereof, and the like; and R^{a} is hydrogen, alkly, alkylsulfonic acid, or alkylsulfonate, and salts thereof; or at least one of R and Ra the atoms to which they are attached form a heterocycle; and, in another embodiment,

[chemical structure]

where X is oxygen, nitrogen, or sulfur, and where X is attached to linker L; and each R is independently selected in each instance from H, alkyl, heteroalkyl, and the like; and n is an integer from 0 to about 4; and in another illustrative embodiment,

[chemical structure]

wherein R_{A} and R_{B} are independently selected in each instance from alkyl, heteroalkyl, alkylsulfonic acid, alkylsulfonate, or a salt thereof, or an amine or a derivative thereof; L₁ is a divalent linker attached to the targeting ligand; R is independently selected in each instance from alkyl, heteroalkyl, or alkylsulfonic acid, or alkylsulfonate, or a salt thereof; n is independently in each instance an integer from 0 to about 3; x is an integer from about 1 to about 4; and Het is selected from the group consisting of

[chemical structure]

wherein * is the attachment point; and R_{C} is alkyl or heteroalkyl.

In other embodiments, the ligand-chromophore conjugate as herein described can be selected, for example, from the group consisting of

[chemical structure]

[chemical structure]

[chemical structure]

R represents the following:
PEG(20k) (CH₂CH₂O)₇₆—CH₃
PEG(5k) (CH₂CH₂O)₄₅₄—CH₃
PEG2(60k)

[chemical structure]

Rhodamine PEG Conjugates,

[chemical structure]

[chemical structure]

[chemical structure]

In one embodiment, ligand-chromophore conjugates described herein can be prepared using synthetic procedures described in WO 2008/057437, the contents of which are incorporated by reference herein. Similar conjugates wherein the group L is folate, a folate analog/derivative, or another folate receptor binding ligand are also described in detail in U.S. Pat. No. 5,688,488, incorporated herein by reference. That patent, as well as related U.S. Pat. Nos. 5,416,016 and 5,108,921, each incorporated herein by reference, describe methods and examples for preparing conjugates useful in accordance with the present invention.

In one embodiment, a ligand conjugate comprising a ^{99m}Tc chelating chemical moiety targeted to cells associated with asthma using a vitamin, such as folate, complexed or chelated to ^{99m}Tc through a chelating moiety attached to folate, can be used to image asthma in vivo. An exemplary ligand conjugate, EC20, is described in U.S. Pat. No. 7,128,893, incorporated herein by reference. In one illustrative example, EC20 (^{99m}Tc complex) is provided. EC20 is a ligand conjugate compound of the formula

[chemical structure]

and can be complexed to ^{99m}Tc to image asthma in vivo.

In another embodiment, asthma can be imaged using the ligand conjugate compound of formula

[chemical structure]

wherein R′ is the side chain of an amino acid and wherein the conjugate is complexed to a radionuclide (e.g., ^{99m}Tc) to image asthma in vivo.

In another embodiment, asthma can be imaged using the ligand conjugate compound of formula

[chemical structure]

wherein R′ is the side chain of an amino acid, D is a divalent linker, and n is 0 or 1, and wherein the conjugate is complexed to a radionuclide (e.g., ^{99m}Tc) to image asthma in vivo.

In one embodiment, the ligand conjugate L-X (e.g., EC20) is pyrogen-free. In another embodiment, the ligand conjugate L-X (e.g., EC20) is administered after administration of unlabeled folate to the patient.

In one embodiment, the ligand conjugate is administered to a patient afflicted with asthma, and following a period of time (e.g., from about 0.1 to about 24 hours), the patient is subjected to the external imaging technique enabled by the ligand conjugate.

It is appreciated that in the embodiments described herein, certain aspects of the methods are presented in the alternative, such as selections for any one or more of L or X in the conjugates L-X. It is therefore to be understood that various alternate embodiments of the invention include individual members of those lists, as well as the various subsets of those lists. Each of those combinations are to be understood to be described herein by way of the lists.

In various embodiments, the ligand conjugates described herein may contain one or more chiral centers, or may otherwise be capable of existing as multiple stereoisomers. Accordingly, it is to be understood that the present invention includes pure stereoisomers as well as mixtures of stereoisomers, such as enantiomers, diastereomers, and enantiomerically or diastereomerically enriched mixtures of the ligand conjugates described herein. The ligand conjugates described herein may also be capable of existing as geometric isomers. Accordingly, it is to be understood that the present invention includes pure geometric isomers or mixtures of geometric isomers.

As used herein, the term “alkyl” includes a chain of carbon atoms, which is optionally branched. As used herein, the term “alkylene” includes a divalent chain of carbon atoms, which is optionally branched. As used herein, the term “alkenyl” and “alkynyl” includes a chain of carbon atoms, which is optionally branched, and includes at least one double bond or triple bond, respectively. It is to be understood that alkynyl may also include one or more double bonds. It is to be further understood that alkyl is advantageously of limited length, including C₁-C₂₄, C₁-C₁₂, C₁-C₈, C₁-C₆, and C₁-C₄. It is to be further understood that alkenyl and/or alkynyl may each be advantageously of limited length, including C₂-C₂₄, C₂-C₁₂, C₂-C₈, C₂-C₆, and C₂-C₄. It is appreciated herein that shorter alkyl, alkenyl, and/or alkynyl groups may add less lipophilicity to the compound and accordingly will have different pharmacokinetic behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium.

As used herein, the term “aryl” includes monocyclic and polycyclic aromatic groups, including aromatic carbocyclic and aromatic heterocyclic groups, each of which may be optionally substituted. As used herein, the term “carboaryl” includes aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “amino” includes the group NH₂, alkylamino, and dialkylamino, where the two alkyl groups in dialkylamino may be the same or different, i.e. alkylalkylamino. Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amino and derivatives thereof” includes amino as described herein, and alkylamino, alkenylamino, alkynylamino, heteroalkylamino, heteroalkenylamino, heteroalkynylamino, cycloalkylamino, cycloalkenylamino, cycloheteroalkylamino, cycloheteroalkenylamino, arylamino, arylalkylamino, arylalkenylamino, arylalkynylamino, acylamino, and the like, each of which is optionally substituted. The term “amino derivative” also includes urea, carbamate, and the like.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, nitro, sulfonic acids and derivatives thereof, carboxylic acids and derivatives thereof, and the like.

While certain embodiments of the present invention have been described and/or exemplified herein, it is contemplated that considerable variation and modification thereof are possible. Accordingly, the present invention is not limited to the particular embodiments described and/or exemplified herein.

### EXAMPLES

### Example 1

### Preparation of EC20-^{99m} Tc

EC20-^{99m}Tc was prepared as described (Leamon et al., Bioconjug Chem, 2002, 13(6): 1200-10; incorporated herein by reference). Vials containing lyophilized EC20 were heated at 100° C. for 5 min, after which two mL of a 925 MBq/mL solution of sodium pertechnetate (Cardinal Health) was added and the vial was heated for an additional 15 min. After dilution with the desired volume of saline, mice were injected i.p. with either 400 μL of imaging agent (18.5 MBq, ˜250 nmoles/Kg of EC20) or the same volume of imaging agent supplemented with 100-fold molar excess of free folic acid (to compete for unoccupied folate receptors). Unbound EC20-^{99m}Tc was allowed to clear from the tissues for a period of four hours prior to imaging.

### Example 2

### Imaging

EC20-^{99m}Tc was prepared as described above. In one exemplary imaging embodiment, animals were allowed to clear for a period of 4 hours prior to imaging. Animals were either anesthetized with 3 to 4% isoflurane or euthanized for the imaging procedure. Images were taken in a KODAK Imaging Station In Vivo FX using the following settings. Image acquisition and ROI analyses were performed using KODAK Molecular Imaging software v. 4.5 (Carestream Molecular Imaging).

White Light Imaging:
1. f-stop—22
2. FOV—200×200 mm
3. Emission—White
4. Excitation—Open
5. Exposure time—0.05 seconds
6. Focus—7 mm

Radioimaging:
1. f-stop—0
2. FOV—200×200 mm
3. Emission—Black
4. Excitation—Open
5. Exposure time—60 seconds
6. Focus—7 mm
7. Radioisotopic phosphor screen

X-ray imaging:
1. f-stop—4
2. FOV—200×200 mm
3. Emission—Black
4. Excitation—Open
5. Exposure time—55 seconds
6. Focus—7 mm
7. Radiographic phosphor screen

### Example 3

### FR-β Identified in Lung Macrophages in a Murine Model of Allergic Asthma

To determine if macrophages from the lungs of asthmatic mice express folate receptors, Balb/c mice were immunized against ovalbumin (OVA) and challenged with OVA by intranasal instillation. Eight-week-old virgin female BALB/c mice (Harlan Laboratories) were sensitized by intraperitoneal (i.p.) injection of 0.3 ml solution containing 100 μg of OVA (Sigma-Aldrich) bound to Imject Alum (Pierce) on days 0, 7, and 14, followed by 50 μg OVA intranasal challenge on days 17, 18, and 19. Mice were either injected with folate conjugates as described below or anesthetized or euthanized for further analysis approximately 24 hours after the last OVA challenge. After allowing 24 hours for allergic inflammation to develop, reverse transcription PCR (RT-PCR) was performed on isolated lung tissue to identify expression of FR-β.

RT-PCR and Quantitative Real-Time PCR:

Total RNA was prepared from sorted cells using the RNAeasy kit (Qiagen). cDNA was synthesized using qscript flex cDNA synthesis kit (Quanta Biosciences) or Superscript III reverse-transcriptase and oligo(dT)₂₀ primer (Invitrogen). PCR consisted of a denaturation step at 94° C. for 2.5 minutes, followed by 45 cycles for FR-β (sense primer, gctggaagactgaactaagacagaa; antisense primer, ggagtcttggatgaagtgactctta) and 30 cycles for hypoxanthine phosphoribosyltransferase (HPRT, sense primer, tgaagagctactgtaatgatcagtcaac; antisense primer, agcaagcttgcaaccttaacca). Each cycle consisted of 30 seconds of denaturation at 94° C., 30 seconds of annealing at 60° C., and 45 seconds of extension at 72° C. Quantitative real-time PCR was carried out using TaqMan primer and probe sets for mouse FR-α, FR-β, FR-δ, arginase-1 (Arg1), iNOS, and 18s rRNA (Applied Biosystems). Relative mRNA expression was calculated from the formula, RmE=2-(Ct of gene-Ct of 18s rRNA), where Ct is the threshold cycle time and RmE is the relative mRNA expression. Data were normalized to 18s RNA and are representative of three independent experiments.

FR-β transcripts are significantly increased in murine lung tissue during OVA-induced acute allergic inflammation related to asthma. As shown in FIG. 1A, OVA sensitization induced allergic inflammation in the mice, as demonstrated by a massive increase in eosinophil content in the OVA-treated mice. A significant accumulation of alternatively activated macrophages (AAMs) was also shown by the increase in arginase activity in the same sample (see FIG. 1B). Evidence that at least a portion of the accumulating macrophages express FR-β was then shown by demonstrating an enhancement in FR-β mRNA (see FIG. 1C).

Flow cytometry was performed on all cells obtained by collagenase digestion of asthmatic lungs using an anti-FR polyclonal antibody in order to confirm the distribution of FR-β expression on activated macrophages. FR-β is expressed on F4/80′ macrophages in lungs from mice during OVA-induced acute allergic inflammation related to asthma. As shown in FIG. 2A, 97.2% and 98.5% of FR positive lung cells also stained for F4/80 and CD68, respectively, suggesting that FR expression is limited to monocyte/macrophage lineage cells in the lungs. Furthermore, quantitative RT-PCR (qRT-PCR) analysis of sorted FR⁺F4/80⁺ cells showed that FR-positive macrophages express mRNA for FR-β, but not for FR-α or FR-δ (see FIG. 2B). Moreover, FR-β mRNA could be detected in sorted F4/80⁺ cells, but not in F4/80⁻ cells (see FIG. 2C). This example suggests that expression of FR-β is limited to the macrophage population of cells in asthmatic lungs.

### Example 4

### Fraction of FR-β Positive Macrophages Increases Following Ova-Induced Allergic Inflammation

To better quantify the increase in FR-B⁺ macrophages upon induction of allergic asthma, the fraction of FR-B⁺ macrophages in the lungs of PBS and OVA-sensitized mice was compared using flow cytometry.

Isolation of Immune Cells from the Lungs of Asthmatic Mice:

Euthanized mice were subjected to bronchoalveolar lavage prior resection and mincing of their lung tissue into small cubes. The minced tissue was then digested for 60 minutes in 5 milliliters (ml) of folate-deficient RPMI 1640 media (Life Technologies) containing 0.1% collagenase (Type IV; Sigma-Aldrich), 0.01% hyaluronidase (Sigma-Aldrich) and 0.002% DNase (Sigma-Aldrich). Digested tissue was further disaggregated into single cells by gentle pipetting through a 1 mL pipette tip and then passed through a 40 μm cell strainer (BD Biosciences). After lysis of residual erythrocytes using red blood cell lysis buffer (Sigma-Aldrich), dissociated cells were layered onto 5 ml of Ficoll-Paque™ PLUS (GE Healthcare) lymphocyte separation solution and centrifuged at 500 g for 30 minutes. The mononuclear cells in the middle layer and the granulocytes at the bottom of the tube were collected for flow cytometric analysis.

Flow Cytometry Analysis and Cell Sorting:

Single cell suspensions obtained as described above from asthmatic lungs were pre-incubated with anti-CD16/32 antibodies (eBioscience) to block F, receptors, then treated for 1 h with a 1:100 dilution of polyclonal rabbit anti-FR antibody (FL-257; Santa Cruz Biotechnologies) followed by staining for 1 hour with a 1:100 dilution of FITC-conjugated anti-rabbit second antibody (Sigma-Aldrich). Evaluation of the isolated cells for functional FR was performed by incubation with folate-Oregon Green (FOG) or folate-AlexaFluor 647 in the presence and absence of 1000-fold excess folic acid to block unoccupied FR. For characterization of co-expression of FR with inducible nitric oxide synthase (iNOS) or mannose receptor (MR), FOG (200 nM) and PE-conjugated anti-F4/80 (eBioscience) labeled cells were fixed, permeabilized, and incubated with FITC-labeled anti-iNOS (BD Biosciences) or AlexaFluor 647-labeled anti-MR (Biolegend). To distinguish FR negative from FR positive cells, the fluorescence gate for FR expression was set such that <1% of the macrophages were counted as FR⁺ in the competition control. Similarly, the fluorescence gate for other cell markers was set such that <1% of the gated cell population appeared positive when examined with a nonspecific antibody isotype control. Experiments from each group were repeated at least 3 times. Flow cytometry and cell sorting were performed on a BD FACSCalibur flow cytometer (BD Biosciences) and iCyt Reflection Sorter (iCyt), respectively. Data were analyzed using CellQuest (BD Biosciences).

F4/80⁺ cells expressing FR-β increase markedly in lung tissue during OVA-induced asthma in mice. As shown FIG. 3A, FR-F⁺ macrophages (F4/80⁺ cells) increased nearly 5-fold (from approximately 8% to 39% of the total macrophage population) following induction of allergic asthma. Moreover, the fraction of total lung cells that were macrophages (F4/80⁺) increased from 6% to 19%. Thus, the fraction of lung macrophages that express FR-β increased approximately 15-fold in OVA-treated mice compared to PBS-treated mice. Consistent with these observations, the total number of FR-β⁺ macrophages per lung increased from 2.6×10⁴ to 8.7×10⁵ upon induction of the allergic inflammation (see FIG. 3B). After adjustment for the nearly 70% increase in lung weight in the asthmatic mice, these results demonstrate an approximate 20-fold rise in FR-B⁺ macrophages per gram of lung tissue upon induction of allergic inflammation.

### Example 5

### FR-β Receptor Expressed on Asthmatic Lung Macrophages is Functional

The ability of FR-β to bind folic acid (FA) conjugates is essential for use of the receptor as a mediator of FA-conjugated drug delivery. The FR-β receptor upregulated in asthmatic lungs was analyzed to determine if the receptor is functional for binding folic acid (FA) conjugates. Harvested lung mononuclear cells were incubated with folate-Oregon Green (FOG) and anti-F4/80 for evaluation using flow cytometry. Flow cytometry was performed according to the methods described in Example 4.

As shown in FIG. 4A, a subpopulation of lung macrophages (F4/80⁺ cells) from asthmatic mice bound high levels of FOG. Moreover, the binding of FOG was inhibited by a 1000-fold molar excess of free folic acid, demonstrating that cell binding of FOG was FR-β mediated.

To further verify the functionality of FR-β on asthmatic macrophages in vivo, folate-DyLight 680 was injected intravenously (i.v.) into mice that had been previously immunized and challenged with OVA.

In Vivo Near-Infrared Fluorescent Imaging:

Mice were injected via the tail vein with 10 nmol of folate-DyLight™ 680 (prepared in our lab) in 50 μl of saline. After a 2 hour clearance period, mice were anesthetized with 3% isoflurane, and near-infrared fluorescence imaging was performed using the IVIS® Lumina II Imaging System (Caliper Life Sciences). For fluorescence acquisition, a 692 nm excitation filter and a 712 nm emission filter were used. Abdomens were shielded with black construction paper to avoid interference from fluorescence emanating from the kidneys and bladder. After in vivo imaging, animals were euthanized by CO₂ aphyxiation and lungs were resected for further imaging.

Two hours after folate-DyLight 680 injection, the lungs of the mice were resected and imaged ex vivo for folate conjugate uptake. For histopathologic assessment, lungs of mice were excised, fixed in IHC zinc fixative solution (BD Pharmingen) and embedded in paraffin. Sections of fixed tissues (4 pm thick) were prepared and stained with hematoxylin and eosin (H&E) for light microscopic examinations

As shown in the representative samples of FIG. 4B, lungs from the OVA-treated mice were brightly fluorescent, whereas lungs from PBS-treated mice were essentially nonfluorescent. Furthermore, as shown in FIG. 4B (right panel), the binding of folate-DyLight680 to asthmatic lungs was FR-β specific because uptake of the folate-targeted dye was readily inhibited by 100-fold excess of folate-gluocosamine (i.e., a ligand that associates with FR-β with high affinity).

### Example 6

### FR-β is a Marker for Alternatively Activated Macrophages in Lungs of Mice Induced to Develop Allergic Asthma

Previous studies show that human peripheral blood monocytes can be differentiated in vitro into FR-β expressing alternatively activated macrophages (AAMs) by repeated treatment with M-CSF. Because AAMs may participate intrinsically in the development of asthma, the characteristics of FR-β-positive macrophages present in the lungs of asthmatic mice were examined. FIG. 5A shows that AAMs from evaluated mice do not express iNOS, a marker of classically activated macrophages (CAMs). However, as shown in FIG. 5B, virtually all FR-β⁺ macrophages express the mannose receptor (MR), a hallmark of AAMs. In agreement with the flow cytometry data, mRNA expression of Arg1, another AAMs marker, is also elevated in the FR-β-positive macrophage fraction (see FIG. 5C). These qRT-PCR data were confirmed by analysis of arginase catalytic activity in the macrophage populations of naïve and asthmatic mice (see FIG. 5D).

Arginase Activity Assay:

For analysis of arginase (Arg1) enzyme function, sorted cells from freshly isolated lungs were resuspended at a concentration of 1×10⁷ cells/mL in lysis buffer. Arg1 activity was measured in supernatants of cell lysates using the QuantiChrom™ Arginase Assay Kit (DARG-200, BioAssay Systems). Protein concentration was determined using BCA Protein Assay Kit (Pierce Biotechnology Inc.).

### Example 7

### Folate-Targeted Radioimaging Agents can Assess the Activation Status of Lung Macrophages in Asthmatic Mice In Vivo

As shown in Example 4, FR-3-positive macrophages are virtually absent from lungs of healthy mice. Thus, radioimaging agents were evaluated for their ability to noninvasivey assess the activation status of pulmonary macrophages in asthmatic mice in vivo. In this example, a folate-targeted ^{99m}Tc-based radioimaging agent (^{99m}Tc-EC20) was injected into mice with OVA-induced asthma. Thereafter, γ-scintigraphic and SPECT/CT images were obtained.

Preparation of ^{99m}Tc-EC20:

Vials containing formulated EC20, a folate-targeted technetium (^{99m}Tc) radioimaging agent, were obtained from Endocyte (West Lafayette, IN). EC20 was reacted with ^{99m}Tc by submersing in a boiling water bath, allowing to heat for 5 minutes, and then injecting with 2 mL of a 925 MBq/mL solution of ^{99m}Tc (Cardinal Health). After heating for an additional 15 minutes, the folate-targeted radioimaging agent was diluted with the desired volume of saline, and mice were injected i.p. with either 400 μL of ^{99m}Tc-EC20 (18.5 MBq, ˜250 nmol of EC20 per kilogram) or the same volume of ^{99m}Tc-EC20 plus with a 100-fold molar excess of free folic acid (to compete for unoccupied FRs). Unbound ^{99m}Tc-EC20 was allowed to clear from the tissues for 4 hours before imaging.

Radioimaging and Assessment of ^{99m}Tc-EC20 Accumulation:

Mice injected with ^{99m}Tc-EC20 were anesthetized with 3% isoflurane and imaged using a Kodak Image Station (Carestream Molecular Imaging). Radioimage acquisition was performed for 2 minutes (whole body) and 15 minutes (resected lungs) using a radioisotopic phosphor screen (Carestream Molecular Imaging), no illumination source, a 4×4 binning setting, and an f-stop of 0. Radiographic images used to coregister anatomic structures with γ-scintigraphic images during overlays were acquired for 55 seconds using the same Kodak Imaging Station. Both radiographic and γ-scintigraphic images had a focus setting of 7 mm and a field of view of 200×200 mm. The 7-emission from the abdomen was shielded using a 5-mm-thick lead shield. All data were analyzed using the Kodak molecular imaging software (version 4.5; Carestream Molecular Imaging). Single-photon-emission computed tomography/computed tomography (SPECT/CT) scans were performed using MILab's sub-half-mm resolution U-SPECT-II/CT scanner (Utrecht, The Netherlands). Before scanning, kidneys and livers were removed from the mice to reduce noise from radioactivity accumulation in those organs in the field of view. Acquisition time was 60 minutes for SPECT (30 min/frame, 2 frames) and 30 minutes for CT (‘normal’ acquisition at 55 kV and 500 μA). SPECT and CT reconstruction was performed using a POSEM algorithm and a cone-beam filtered back-projection algorithm (NRecon v1.6.3, Skyscan), respectively. Co-registered images were visualized using the PMOD software (PMOD Technologies, Zurich, Switzerland). For quantitation of accumulation of ^{99m}Tc-EC20 in the lungs, mice were euthanized by CO₂ asphyxiation, lungs were excised, and radioactivities were counted for 2 minutes using a γ-counter (Packard Bioscience). Results are reported as percentage injected dose per gram of tissue (% ID/g).

As shown in FIG. 6A, healthy mice showed no pulmonary uptake of ^{99m}Tc-EC20. In contrast, asthmatic mice displayed prominent uptake of ^{99m}Tc-EC20 in multiple loci throughout the affected lungs (see FIG. 6B). The elevated lung accumulation was a result of FR-β expressing cells, as demonstrated by the absence of uptake in mice pre-injected with 100-fold excess of folate-glucosamine (i.e., a high affinity FR-β ligand that can block empty folate receptors).

Further confirmation of the in vivo imaging data was also obtained from ex vivo γ-scintigraphic images of lungs resected from the treated mice (see FIG. 6C). These scintigraphic images were confirmed by gamma counting of the same tissues (see FIG. 6D). Taken together, these data demonstrate that uptake of folate conjugates is present in asthmatic lungs, but not in healthy lungs. Thus, folate-targeted imaging agents can evaluate the inflammatory status of asthma patients.

What is claimed is:

1. A method of imaging asthma, said method comprising the steps of:
administering to a patient afflicted with asthma an effective amount of a composition comprising a conjugate of the general formula
L-X
wherein the group L comprises a ligand, wherein the ligand is a folate, and wherein the group X comprises a chromophore capable of emitting light; and
imaging the asthma.

2. The method of claim 1 wherein the chromophore is selected from the group consisting of a fluorophore, a Raman enhancing dye, an hematoporphyrin, and derivatives thereof.

3. The method of claim 2 wherein the chromophore is a fluorophore.

4. The method of claim 1 wherein the chromophore is selected from the group consisting of a fluorescein, a rhodamine, a cyanine, a DyLight Fluor, and an Alexa Fluor.

5. The method of claim 1 wherein the chromophore has the formula [chemical structure]
where X is oxygen, nitrogen, sulfur, S(O)₂, or C(O), and where X is attached via a divalent linker to the ligand; Y is OR^{a}, NR^{a}₂, or NR^{a}₃⁺; and Y′ is O, NR^{a}, or NR^{a}₂⁺; n is in each instance independently selected from 0, 1, 2, or 3; where each R is independently selected in each instance from H, alkyl, alkyloxy, heteroalkyl, fluoro, sulfonic acid, sulfonate, and salts thereof; and R^{a} is hydrogen, alkly, alkylsulfonic acid, or alkylsulfonate, and salts thereof; or at least one of R and Ra the atoms to which they are attached form a heterocycle.

6. The method of claim 1 wherein the chromophore has the formula [chemical structure]
where X is oxygen, nitrogen, or sulfur, and where X is attached via a divalent linker to the ligand; and each R is independently selected in each instance from hydrogen, alkyl, heteroalkyl; and n is an integer from 0 to about 4.

7. The method of claim 1 wherein the chromophore has the formula [chemical structure]
wherein R_{A} and R_{B} are independently selected in each instance from alkyl, heteroalkyl, alkylsulfonic acid, alkylsulfonate, or a salt thereof, or an amine or a derivative thereof; L₁ is an alkylene linked via a divalent linker to the ligand; R is independently selected in each instance from alkyl, heteroalkyl, or alkylsulfonic acid, or alkylsulfonate, or a salt thereof; n is independently in each instance an integer from 0 to about 3; x is an integer from about 1 to about 4; and Het is selected from the group consisting of [chemical structure]
wherein * is the attachment point; and R_{C} is alkyl or heteroalkyl.

8. The method of claim 1 wherein the chromophore is selected from the group consisting of Cy3, Cy5, Cy7, Oregon Green 488, Oregon Green 514, AlexaFluor 488, AlexaFluor 647, tetramethylrhodamine, DyLight 680, CW 800, and Texas Red.

9. The method of claim 1 wherein the chromophore is fluorescein.

10. The method of claim 1 wherein the folate has the formula [chemical structure]
wherein Y¹ and Y² are each-independently selected from the group consisting of halo, R², OR², SR³, and NR⁴R⁵;
U, V, and W represent divalent moieties each independently selected from the group consisting of —(R^{6a})C═, —N═, —(R^{6a})C(R^{7a})—, and —N(R⁴)—; Q is selected from the group consisting of C and CH; T is selected from the group consisting of S, O, N, and —C═C—;
A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;
R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;
R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;
D is a divalent linker;
* represents the attachment point for X; and
n, p, r, s and t are each independently either 0 or 1.

11. The method of claim 1 wherein the folate has the formula [chemical structure]
wherein * indicates the attachment point to a divalent linker attached to the chromophore.

12. A method of imaging asthma, said method comprising the steps of:
administering to a patient afflicted with asthma an effective amount of a composition comprising a conjugate of the general formula
L-X
wherein the group L comprises a ligand, wherein the ligand is a folate, and wherein the group X comprises a chemical moiety that emits radiation; and
imaging the asthma.

13. The method of claim 12 wherein the group X comprises a metal chelating moiety that chelates a metal cation.

14. The method of claim 13 wherein the metal cation is a radionuclide.

15. The method of claim 14 wherein the radionuclide is ^{99m}Tc.

16. The method of claim 13 wherein the metal cation is a nuclear magnetic resonance imaging enhancing agent.

17. The method of claim 12 wherein the folate has the formula [chemical structure]
wherein Y¹ and Y² are each-independently selected from the group consisting of halo, R², OR², SR³, and NR⁴R⁵;
U, V, and W represent divalent moieties each independently selected from the group consisting of —(R^{6a})C═, —N═, —(R^{6a})C(R^{7a})—, and —N(R^{4a}s)—; Q is selected from the group consisting of C and CH; T is selected from the group consisting of S, O, N, and —C≡C—;
A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;
R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;
R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;
D is a divalent linker;
* represents the attachment point for X; and
n, p, r, s and t are each independently either 0 or 1.

18. The method of claim 12 wherein the conjugate comprises a compound of the formula [chemical structure]
wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein the compound is bound to a radionuclide.

19. The method of claim 12 wherein the conjugate has the formula [chemical structure]
wherein the conjugate is bound to an isotope of technicium and wherein the isotope of technicium is ^{99m}Tc.

20. The method of claim 12 wherein the folate has the formula [chemical structure]
wherein * indicates the attachment point to a divalent linker attached to the group X.
